(19) United States
(12) Patent Application Publication
(10) Pub. No.: US 2007/0123594 A1
(43) Pub. Date: May 31, 2007

(54) Titania supports for fisher-tropsch catalysts

(76) Inventors: Ronald Jan Dogterom, Amsterdam (NL); Marinus Johannes Reynhout, Amsterdam (NL)

Correspondence Address: SHELL OIL COMPANY, P O BOX 2463, HOUSTON, TX, 772522463, US

(21) Appl. No.: 10/573,795

(22) PCT Filed: Sep. 30, 2004
(86) PCT No.: PCT/EP04/52379

§ 371 Date: Mar. 28, 2006

(30) Foreign Application Priority Data
Sep. 30, 2003 (EP) 03103612.2

(51) Int. Cl. C07C 27/06

(52) U.S. Cl. 518/716

(57) ABSTRACT

A shaped catalyst carrier containing titania, wherein at least 50 wt % of the titania is present as brookite, and wherein the carrier has between 40 and 100 wt % of crystalline titania based on the total weight of the carrier. Also disclosed are catalyst or catalyst precursors formed from the shaped catalyst carriers described above, and a Group VIII metal or metal compound, and their use in the synthesis of hydrocarbons from carbon monoxide and hydrogen.

### FIELD OF THE INVENTION

The invention relates to shaped titania catalyst carriers, catalyst precursors or catalysts, and to a process for the preparation of hydrocarbons from synthesis gas using the new catalysts.

### BACKGROUND OF THE INVENTION

The use of titania (or titanium dioxide) as white inorganic pigment is well known. Two processes are used to prepare titania on a commercial scale, namely the so-called “chloride process” and the “sulphate process”. See for instance Ullmann's Encyclopedia of Industrial Chemistry, Fifth edition, Vol. A20, pages 271-281.

Beside the use of titania as a pigment, there are also other applications. One other application of titania is the use as catalyst carrier. The uses and performances for a given catalyst application are, however, strongly influenced by the crystalline structure, the morphology and the size of the particles. Nanosized TiO₂ particles are of particular interest because of their specifically size-related properties.

Polymorphs of titania that occur naturally are rutile, anatase, and brookite. Nanosized titania, obtained when prepared via a commercial process, is commonly anatase. When using the chloride process, up to 20 wt % rutile can be formed. Brookite is sometimes formed in small quantities as contaminant. Anatase transforms to rutile under certain conditions, such as high calcination temperatures, since it is the more stable polymorph of titania.

The preparation of hydrocarbons from a gaseous mixture comprising carbon monoxide and hydrogen (synthesis gas) by contacting the mixture with a catalyst at elevated temperature and pressure is known as the Fischer-Tropsch synthesis. Catalysts used in the Fischer-Tropsch synthesis often comprise a titania based support material and one or more metals from Group VIII of the Periodic Table of Elements, especially from the iron group, optionally in combination with one or more metal oxides and/or metals as promoters. Particular interest has been given to catalysts comprising cobalt as the catalytically active component, in combination with one or more promoters selected from zirconium, titanium, chromium, vanadium and manganese, especially manganese, and supported on a titania carrier. Such catalysts are known in the art and have been described, for example in the specifications of International patent application publication No. WO-A-9700231, United States patent publication No. U.S. Pat. No. 4,595,703 and European Patent Applications No. 96203538.2 and 96202524.3. U.S. Pat. No. 4,595,703 describes titania based catalysts having various rutile:anatase ratios.

At the conditions reached in commercial practice, the Fischer-Tropsch reaction yields almost equal amounts of water and paraffins on weight base. Consequently, the catalyst is being exposed to large amounts of steam at elevated temperatures, which has been shown to influence the performance of cobalt catalysts in a variety of ways, for example it may result in a decrease of activity and/or selectivity and consequently, in catalyst lifetime. One of the unwanted reactions is the formation of CoTiO₃, which is difficult to reduce under Fischer-Tropsch conditions and even under the usual regeneration conditions. Furthermore, water vapor causes the irreversible transformation of anatase to relatively large particles of rutile, resulting in a decrease in the surface area of the catalyst and a deactivation of the catalyst.

To improve the resistance of the catalyst against water vapour, several solutions can be applied, mainly focussed on the modification of the anatase support. From literature it is known that the (hydro-) thermal stability of anatase can be improved by modification of the anatase crystals by means of dopants such as SiO₂, ZrO₂ and Al₂O₃. These homogeneous multi-component oxides show higher anatase-rutile transition temperatures and thus an improved stability of the anatase crystals. Depending on its concentration, also manganese is a well-known anatase stabiliser. At low concentration, especially below 1.5 mol %, manganese ions are incorporated in the TiO₂ structure and the anatase phase is stabilised, but at higher concentrations part of the manganese is segregated on the surface and the rutile formation is accelerated.

Application of the polymorph rutile, which is the most thermodynamically stable TiO₂ form, seems the most promising route. However, it is difficult to synthesise nanosized rutile particles, which is a requirement for obtaining homogeneous and good dispersed cobalt catalysts. Furthermore, the rutile particles are difficult to shape into a suitable catalyst support. The object of the present invention is to provide a hydro-thermally stable shaped titania support, which does not require the use of dopants.

### SUMMARY OF THE INVENTION

It has now been found that the above can be, achieved by using titania as shaped catalyst carrier, wherein at least 50 wt % of the crystalline titania is present as brookite, and wherein the carrier comprises between 40 and 100% crystalline titania based on the total weight of the carrier, preferably between 70 and 100 wt %. It has particular been found that the shaped catalyst carrier is very suitable for the preparation of catalysts or catalyst precursors comprising a Group VIII metal or a Group VIII metal compound. It has furthermore been found that the addition of binder materials results in even more stronger shaped catalyst, the binder suitably being an inorganic binder, e.g. a refractory oxide binder, the binder not being a continues polymeric organic matrix binder, e.g. a continuous polymer matrix of a polyolefin. It has moreover been found that the catalyst is very suitable for the preparation of hydrocarbons comprising contacting a mixture of carbon monoxide and hydrogen after activation by reduction with hydrogen at elevated temperature.

### DETAILED DESCRIPTION OF THE INVENTION

Applicants have found that when titania is used wherein at least 50 wt % brookite is present, the catalyst or catalyst precursor comprising a Group VIII metal or metal compound, prepared with the brookite containing shaped catalyst carrier, has an improved hydrothermal stability when used under the Fischer-Tropsch reaction conditions. An additional advantage is that shaping of the material results in mechanically stronger shaped catalyst carrier than using titania in the anatase or rutile form. The term ‘shaped’ relates to a process wherein catalyst particles are formed from a powder, each of particles having a particular shape. Suitable processes are spray drying, pelletizing, (wheel) pressing and extrusion, preferably spray drying and extrusion. Coating processes, e.g. spray coating, dip-coating and painting are not included. The volume of the shaped catalyst carrier particles is suitably between 1 and 250 mm³, preferably between 2 and 100 mm³, more preferably between 4 and 50 mm³, especially in the case that an extrusion process is used. In the special case that spray drying is used the average particle size is suitably between 5 and 500 micron, preferably between 10 and 200 micron.

The shaped catalyst support preferably comprises of between 70 and 100 wt % of crystalline titania. The non-titania component may be, for example, a binder as described below.

The titania suitably has at least 60 wt % present as brookite. The titania may be present up to 100% as brookite, preferably at most 90 wt % present as brookite and more preferably at most 80 wt %. The presence and content of brookite can be distinguished from other morphologies by using X-ray diffraction analysis, as for example, described in the article of A. Pottier cited here below.

The titania may furthermore comprise rutile, preferably in the range of from 0 to 50 wt %, more preferably in the range of from 5 to 30 wt %. The titania may also comprise anatase, preferably in the range of from 0 to 10 wt %, more preferably in the range of from 0 to 5 wt %. The total of the three polymorphs brookite, rutile and anatase will typically amount to 100% of the titania present in the shaped catalyst carrier. In addition to the crystalline titania, a certain amount of amorphous titania may be present. Suitably this amount is less than 50 wt % based on the total weight of titania, preferably less than 20 wt %, more preferably less than 5 wt %.

The primary size of the brookite particles can be determined using XRD line broadening techniques as described in, for example, the cited article of A. Pottier here below and or high-resolution transmission electron microscopy (HRTEM). Also the morphology of the brookite particles can be determined using HRTEM. Depending on the preparation conditions, different morphologies of brookite particles are obtained, namely spheroidal particles or platelets. The specific surface area is, among others, determined by the size of the primary particles. The size of the primary particle of brookite is preferably in the range of from 10 to 100 nm, more preferably of from 20 to 70 nm. Preferably the brookite nanoparticles are obtained by addition of TiCl₄ to an aqueous solution of hydrochloric acid or perchloric acid of at least 2 M wherein brookite particles are formed by thermolysis. The brookite particles are isolated, after washing, by well known techniques such as centrifuge, filtration, drying and the like. A suitable example of such a method to prepare nano-sized brookite polymorph of titania according to this invention is described by A Pottier et al., J. Mater. Chem. 11 (2001) 1116-1121, which publication is hereby incorporated by reference.

Methods of preparing a shaped catalyst carrier include spray drying, pressing, extruding or otherwise forcing a granular or powdered catalyst or catalyst precursor material into various shapes under certain conditions, which will ensure that the particle retains the resulting shape, both during reaction as well as during regeneration. The preferred method for preparing a shaped catalyst carrier according to our invention is by extrusion, especially if the catalyst is to be applied in a fixed bed reactor. If the catalyst is to be used in a slurry reactor the shaped catalyst carriers are preferably prepared by spray drying.

The brookite nano-particles are suitable to make a strong shaped catalyst without the requirement of adding a substantial amount of additional binder material. Nevertheless it is advantageous to add such binder materials in order to make an even more stronger shaped catalyst. The shaped catalyst carrier may suitably comprise up to 30 wt % of another refractory oxide, typically amorphous silica, alumina, zirconia or titania, organic glues, a clay or combinations thereof as a binder material, preferably up to 20% by weight based on the total weight of titania and binder material. More preferably a silica and alumina mixture is used as binder where the binder makes up less than about 30 wt %, preferably less than about 20 wt %, more preferably about 3-20 wt %, still more preferably 4-15 wt %, yet more preferred 5-10 wt % of the total shaped catalyst support. The silica and alumina binder mixture may contain 50 wt % or less silica, preferably about 3-50 wt % silica, more preferably 5-35 wt % silica. In order to achieve the benefits of porosity and strength, binder components are mixed with the titania starting material before the shaping operation. They may be added in a variety of forms, as salts or preferably as colloidal suspensions or sols. For example, alumina sols made from aluminium chloride, acetate, or nitrate are preferred sources of the alumina component. Readily available silica sols are preferred sources of the silica component. In each case, however, care must be taken to avoid contamination of these binder sols by elements that are harmful to the active Fischer-Tropsch metals. For example, alkali and alkaline earth cations and sulfur-containing anions such as sulfate are potent poisons of cobalt under Fischer-Tropsch conditions, and hence must be minimized in preparing supports for, for example, cobalt catalysts.

The shaped catalyst carrier, optionally after calcination, may very suitably be used for the preparation of catalysts, especially catalysts suitable for the preparation of hydrocarbons from synthesis gas, a reaction which is known in the literature as the Fischer-Tropsch reaction. The shaped catalyst carrier after calcination show a surface area between 20 and 180 m²/g (BET), preferably between 30 and 120 m²/g. The pore volume is usually between 0.15 and 0.50 cc/g (N₂/77K), preferably between 0.20 and 0.40 cc/g.

Catalysts for use in this process frequently comprise, as the catalytically active component, a metal from Group VIII of the Periodic Table of Elements. Particular catalytically active metals include ruthenium, iron, cobalt and nickel. Cobalt is a preferred catalytically active metal.

The catalyst comprises the shaped catalyst carrier as described above and an amount of catalytically active metal. The content of metal is preferably in the range of from 3 to 75 wt % relative to the total amount of the catalyst, more preferably from 10 to 45 wt %, especially from 15 to 40 wt %.

If desired, the catalyst may also comprise one or more metals or metal oxides as promoters. Suitable metal oxide promoters may be selected from Groups IIa, IIIb, IVb, Vb and VIb of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are very suitable promoters. Particularly preferred metal oxide promoters for the catalyst used to prepare the catalysts for use in the present invention are manganese and zirconium oxide. Suitable metal promoters may be selected from Groups VIIb or VIII of the Periodic Table. Rhenium and Group VIII noble metals are particularly suitable, with rhenium, platinum and palladium being especially preferred. The amount of promoter present in the catalyst is suitably in the range of from 0.01 to 50 wt %, preferably 0.1 to 30 wt %, more preferably 0.5 to 20 wt %, relative to the total amount of the catalyst. The most preferred promoters are selected from vanadium, manganese, rhenium, zirconium and platinum. Preferred metal combinations are CoMn, CoV, CoRe, CoPt or CoPd.

The catalytically active metal and the promoter, if present, may be deposited on the shaped carrier according to the invention by any suitable treatment, such as impregnation and deposition precipitation. Preferably the catalyst is prepared by mixing a source of brookite particles and a source of catalytically active metal and optionally metal promoters, kneading the mixture and extruding the mixture to obtain a shaped catalyst precursor after the typical drying and, optionally, calcinations steps.

If the catalyst is to be used in a so-called slurry Fischer-Tropsch process the catalysts are preferably made by incipient wetness impregnation of spray-dried supports.

A kneading/mulling method for the preparation of the catalyst or a catalyst precursor according to the present invention using titania as catalyst carrier can be performed comprising the following steps: (a) mixing (1) titania in which at least 50 wt % of the crystalline titania is present as brookite, (2) a liquid, and (3) a Group VIII containing compound, which is at least partially insoluble in the amount of liquid used, to form a mixture, (b) shaping and drying of the mixture thus-obtained, and (c) calcination of the mixture thus-obtained.

The liquid may be any of suitable liquids known in the art, for example water; ammonia; alcohols, such as methanol, ethanol and propanol; ketones, such as acetone; aldehydes, such as propanal and aromatic solvents, such as toluene. A most convenient and preferred liquid is water.

Typically, the ingredients of the mixture are mulled for a period of from 5 to 120 minutes, preferably from 15 to 90 minutes. During the mulling process, energy is put into the mixture by the mulling apparatus. The mulling process may be carried out over a broad range of temperature, preferably from 15 to 90° C. As a result of the energy input into the mixture during the mulling process, there will be a rise in temperature of the mixture during mulling. The mulling process is conveniently carried out at ambient pressure. Any suitable, commercially available mulling machine may be employed. The amount of energy used in the mulling process is suitably between 0.05 and 50 Wh/min/kg, preferably between 0.5 and 10 Wh/min/kg.

To improve the flow properties of the mixture, it is preferred to include one or more flow improving agents and/or extrusion aids in the mixture prior to extrusion. Suitable additives for inclusion in the mixture include fatty amines, quaternary ammonium compounds, polyvinyl pyridine, sulphoxonium, sulphonium, phosphonium and iodonium compounds, alkylated aromatic compounds, acyclic mono-carboxylic acids, fatty acids, sulphonated aromatic compounds, alcohol sulphates, ether alcohol sulphates, sulphated fats and oils, phosphonic acid salts, polyoxyethylene alkylphenols, polyoxyethylene alcohols, polyoxyethylene alkylamines, polyoxyethylene alkylamides, polyacrylamides, polyols and acetylenic glycols. Preferred additives are sold under the trademarks Nalco and Superfloc.

To obtain strong extrudates, it is preferred to include in the mixture, prior to extrusion, at least one compound which acts as a peptising agent for the titania. Suitable peptising agents for inclusion in the extrudable mixture are well known in the art and include basic and acidic compounds. Examples of basic compounds are ammonia, ammonia-releasing compounds, ammonium compounds or organic amines. Such basic compounds are removed upon calcination and are not retained in the extrudates to impair the catalytic performance of the final product. Preferred basic compounds are organic amines or ammonium compounds. A most suitable organic amine is ethanol amine. Suitable acidic peptising agents include weak acids, for example formic acid, acetic acid, citric acid, oxalic acid, and propionic acid.

Optionally, burn-out materials may be included in the mixture, prior to extrusion, in order to create macropores in the resulting extrudates. Suitable burn-out materials are commonly known in the art.

The total amount of flow-improving agents/extrusion aids, peptising agents, and burn-out materials in the mixture preferably is in the range of from 0.1 to 20% by weight, more preferably from 0.5 to 10% by weight, on the basis of the total weight of the mixture. Examples of suitable catalyst preparation methods as described above are disclosed in WO-A-9934917.

The Group VIII is preferably a cobalt containing compound. Any cobalt compound of which at least 50% by weight is insoluble in the amount of the liquid used, can be suitably used in the kneading/mulling method of the present invention. Preferably, at least 70% by weight of the cobalt compound is insoluble in the amount of liquid used, more preferably at least 80% by weight, still more preferably at least 90% by weight. Examples of suitable cobalt compounds are metallic cobalt powder, cobalt hydroxide, cobalt oxide or mixtures thereof, preferred cobalt compounds are Co(OH)₂ or Co₃O₄.

The amount of cobalt compound present in the mixture may vary widely. Typically, the mixture comprises up to 40 wt % cobalt relative to the total amount of catalyst, preferably 10-30 wt %. The above amounts of cobalt refer to the total amount of cobalt, on the basis of cobalt metal, and can be determined by known elemental analysis techniques, e.g. SEM or TEM. The cobalt compound may further comprise a Group IVb and/or a Group VIIb compound, preferably a zirconium, manganese or rhenium compound. The most preferred cobalt-containing compound is a mixed cobalt manganese hydroxide.

The cobalt compound which is at least partially insoluble in the liquid may be obtained by precipitation. Any precipitation method known in the art may be used. Preferably, the cobalt compound is precipitated by addition of a base or a base-releasing compound to a solution of a soluble cobalt compound, for example by the addition of sodium hydroxide, potassium hydroxide, ammonia, urea, or ammonium carbonate. Any suitable soluble cobalt compound may be used, preferably cobalt nitrate, cobalt sulphate or cobalt acetate, more preferably cobalt nitrate. Alternatively, the cobalt compound may be precipitated by the addition of an acid or an acid-releasing compound to a cobalt ammonia complex. The precipitated cobalt compound may be separated from the solution, washed, dried, and, optionally, calcined. Suitable separation, washing, drying and calcining methods are commonly known in the art.

In one embodiment of the process of the present invention, the cobalt compound and the compound of promoter metal are obtained by co-precipitation, most preferably by co-precipitation at constant pH. Co-precipitation at constant pH may be performed by the controlled addition of a base, a base-releasing compound, an acid or an acid-releasing compound to a solution comprising a soluble cobalt compound and a soluble compound of promoter metal, preferably by the controlled addition of ammonia to an acidic solution of a cobalt compound and a promoter metal compound.

After deposition of the metal and, if appropriate, the promoter on the carrier material, the loaded carrier is typically subjected to drying and calcination. The effect of the calcination treatment is to remove all water, to decompose volatile decomposition products and to convert organic and inorganic compounds to their respective oxides. In the process according to our invention, the calcination is preferably carried out at a temperature in the range of from 400 to 750° C., more preferably of from 500 to 700° C. Drying is suitably carried out at temperatures between 50 and 250° C.

After calcination, the resulting catalyst precursor is preferably activated by contacting the catalyst with hydrogen or a hydrogen-containing gas, typically at temperatures of about 200 to 350° C.

The invention is also directed to a Fischer-Tropsch process wherein the activated catalyst as here described is used to catalyse the Fischer-Tropsch reaction wherein a mixture of carbon-monoxide and hydrogen is converted to a paraffin wax comprising product.

The mixture of carbon monoxide and hydrogen, also referred to as synthesis gas, are prepared from a (hydro)carbonaceous feeds, for example coal, bio-mass, mineral oil fractions and gaseous hydrocarbon sources. Preferred hydrocarbonaceous feeds for the preparation of synthesis gas are natural gas and/or associated gas. As these feedstocks, after partial oxidation and/or steam reforming, usually result in synthesis gas having H₂/CO ratios of about 2, cobalt is a very good Fischer-Tropsch catalyst as the user ratio for this type of catalysts is also about 2 but may be as low as 1.

The catalytic conversion process may be performed under conventional synthesis conditions known in the art. Typically, the catalytic conversion may be effected at a temperature in the range of from 150 to 300° C., preferably from 180 to 260° C. Typical total pressures for the catalytic conversion process are in the range of from 1 to 200 bar absolute, more preferably from 10 to 70 bar absolute. In the catalytic conversion process especially more than 75 wt % of C₅₊, preferably more than 85 wt % C₅₊ hydrocarbons are formed. Depending on the catalyst and the conversion conditions, the amount of heavy wax (C₂₀₊) may be up to 60 wt %, sometimes up to 70 wt %, and sometimes even up till 85 wt %. Preferably a cobalt catalyst is used, a low H₂/CO ratio is used (especially 1.7, or even lower) and a low temperature is used (190-230° C.). To avoid any coke formation, it is preferred to use an H₂/CO ratio of at least 0.3. It is especially preferred to carry out the Fischer-Tropsch reaction under such conditions that the SF-alpha value, for the obtained products having at least 20 carbon atoms, is at least 0.925, preferably at least 0.935, more preferably at least 0.945, even more preferably at least 0.955. The SF-alpha values are derived from the absolute amount of C₂₀ and C₃₀ produced in the process. Preferably the Fischer-Tropsch hydrocarbons stream comprises at least 35 wt % C₃₀₊, preferably 40 wt %, more preferably 50 wt %.

The Fischer-Tropsch process may be a slurry FT process or a fixed bed FT process, especially a multitubular fixed bed, preferably a three phase fluidised bed process.

### EXAMPLES

The invention will be illustrated by the following non-limiting examples.

### Example 1

Synthesis of Brookite

Nanosized brookite TiO₂ particles were synthesized by thermolysis of titanium tetrachloride in hydrochloric acid. The synthesis mixture was prepared by adding 38 ml TiCl₄ drop wise to 1900 ml of a 3 molar HCl solution, while continuously stirring. The final titanium concentration in the synthesis mixture was 0.18 mol/l. The solution was heated and aged at 100° C. for 48 hours, under statically conditions. All syntheses equipment (ex. Nalgene) was carefully cleaned with concentrated HCl and distilled water.

After ageing the upper liquid was decanted. Distilled water was added to the TiO₂ slurry and the pH was adjusted to 8.0 with a 25% solution of NH₃. The TiO₂ flocculated and migrated to the bottom. The upper liquid was decanted. This was repeated three times in total. After the last decanting step distilled water was added to the TiO₂ slurry and 20 ml of a solution of 10% NH₄NO₃ was added. The formed slurry was filtered over a Büchner filter and the formed cake was washed with 4 litres distilled water. A dry brookite containing sample is obtained by drying the filter cake.

Specific surface areas (BET) were determined using a Quantachrome Autosorb-6 apparatus. X-ray diffraction analysis were obtained using a powder diffractometer (Philips PW1800) operating in the reflecting mode with CuKα radiation. The angular domain was between 2θ 20′ and 85°. The XRD-patterns were analysed for phase recognition and Rietveld quantification. Transmission electron microscopy (TEM) pictures were obtained using a JEOL 2010 apparatus. Samples were prepared by grounding in butanol, subsequently dropped onto a copper grid and finally provided with a carbon film. Elemental analysis was carried out on dried powders using the X-ray fluorescence technique.

### Example 2 (Comparative)

Catalyst Preparation

A mixture was prepared containing 2200 g commercially available titania powder (P25 ex. Degussa), 1000 g of prepared CoMn(OH)x co-precipitate (atomic ratio of Mn/Co is 0.05), 900 g of a 5 wt % polyvinyl alcohol solution and a solution consisting of 300 g water and 22 g of an acidic peptizing agent. The mixture was kneaded for 18 minutes. The loss on ignition (LOI) of the mix was 33.0 wt %. The mixture was shaped using a 1-inch Bonnot extruder, supplied with a 1.7 mm trilob plug. The extrudates were dried for 16 hours at 120° C. and calcined for 2 hours at various temperatures.

### Example 3

Catalyst Preparation

A mixture was prepared using brookite as synthesized according to example 1. A mixture was prepared containing 177 g of brookite (dried basis), 86 g of prepared CoMn(OH)x co-precipitate (atomic ratio of Mn/Co is 0.05) and 2 g of an acidic peptizing agent and 123 g water. The mixture was kneaded for 18 minutes. The loss on ignition (LOI) of the mix was 34.9 wt %. The mixture was shaped using a 1-inch Bonnot extruder, supplied with a 1.7 mm trilob plug. The extrudates were dried for 16 hours at 120° C. and calcined for 2 hours at various temperatures.

### Example 4

Thermal Tests

The thermal stability of the catalysts as prepared in the Examples 2 and 3 were measured by performing calcination experiments at temperatures varying from 550° C. to 700° C., with a heating rate of 143°/hr and a dwell time of 2 hours. The samples were analysed using XRD as described in Example 1. The results are presented in Table 1.

It is evident from the results in the table that the catalyst prepared according to the invention has a higher thermal stability than the titania catalyst prepared according to the prior art. The catalyst according to the invention contains very little of the unwanted CoTiO₃ at a calcination temperature of 650° C., while the prior art catalyst contains 48.2% CoTiO₃.

TABLE 1

| | Calc. | | | | | |
|---|---|---|---|---|---|---|
| | temp. | % | % | % | % | % |
| Catalyst | (° C.) | anatase | brookite | rutile | Co₃O₄ | CoTiO₃ |
| Ex. 2 | 550 | 61 | — | 15 | 24 | 0 |
| Ex. 2 | 600 | 53 | — | 22 | 25 | 0 |
| Ex. 2 | 650 | 2 | — | 49 | 1 | 48.2 |
| Ex. 3 | 550 | — | 70.0 | 4.6 | 25.4 | 0 |
| Ex. 3 | 600 | — | 69.2 | 4.9 | 25.9 | 0 |
| Ex. 3 | 650 | — | 67.5 | 5.1 | 25.1 | 2.3 |

### Example 5

Hydrothermal Stability Tests

The hydrothermal stability of the catalysts as prepared in the Examples 2 and 3 and calcined at the different temperatures of the Example 4 were measured by exposing the catalysts to Fischer-Tropsch process water (mixture of mainly water and a minor fraction of organic acids, alcohols and paraffin's with a pH of 3) at a temperature of 250° C. during 1 week. The experiments were performed in a 400 ml stainless steel autoclave. The treated catalysts were filtered, washed with distilled water and dried at 120° C. The samples were analysed using XRD as described in Example 1. The results are presented in Table 2.

The results in the table demonstrate that the catalyst according to the invention is much more stable under these conditions. While the catalyst as prepared according to the art calcined at 550° C. already shows the formation of CoTiO₃, the catalyst according to the present invention still contains Co₃O₄ only.

TABLE 2

| | Calc. | | | | | |
|---|---|---|---|---|---|---|
| | temp. | % | % | % | % | % |
| Catalyst | (° C.) | anatase | brookite | rutile | Co₃O₄ | CoTiO₃ |
| Ex. 2 | 550 | 43 | — | 27.9 | 19 | 10.4 |
| Ex. 3 | 550 | — | 70.6 | 5.9 | 23.5 | 0 |
| Ex. 3 | 600 | — | 70.7 | 6.1 | 23.1 | 0 |
| Ex. 3 | 650 | — | 69.6 | 6.2 | 21.3 | 2.9 |

### Example 6

The catalyst according to Example 2 and 3 were tested for their performance as a Fischer-Tropsch catalyst in a tubular reactor and a similar selectivity and deactivation was observed. It can thus be concluded that the present invention has provided a more thermal stable and hydro-thermal stable catalyst than the prior art Fischer-Tropsch catalyst without encountering any decrease in catalyst activity or selectivity.

1. A shaped catalyst carrier containing crystalline titania, wherein at least 50 wt % of the crystalline titania is present as brookite and wherein the carrier comprises between 40 and 100 wt % of crystalline titania based on the total weight of the carrier, the shaped catalyst carrier not being a coated catalyst carrier.

2. The carrier of claim 1, wherein at least 60 wt % of the crystalline titania is present as brookite.

3. The carrier of claim 1, wherein at most 90 wt % of the crystalline titania is present as brookite.

4. The carrier of claim 1, wherein the crystalline titania is present as rutile in the range of from 0 to 50 wt %, and wherein the crystalline titania is present as anatase in the range of from 0 to 10 wt %.

5. The carrier of claim 1, wherein the primary particle size of the brookite is in the range of from 10 to 100 nm.

6. The carrier of claim 1, further comprising a binder, and wherein the binder forms from 0 to 20 wt % of the carrier.

7. (canceled)

8. (canceled)

9. (canceled)

10. (canceled)

11. (canceled)

12. (canceled)

13. (canceled)

14. (canceled)

15. A catalyst or catalyst precursor, comprising a Group VIII metal or a Group VIII metal compound and a shaped carrier containing crystalline titania, wherein at least 50 wt % of the crystalline titania is present as brookite and wherein the carrier comprises between 40 and 100 wt % of crystalline titania based on the total weight of the carrier, the shaped catalyst carrier not being a coated catalyst carrier.

16. The catalyst or catalyst precursor of claim 15, wherein the Group VIII metal or Group VIII metal compound is selected from the group consisting of Ru, Fe, Co and Ni.

17. The catalyst or catalyst precursor of claim 15, wherein the Group VIII metal or Group VIII metal compound comprises Co.

18. The catalyst or catalyst precursor of claim 15, further comprising one or more metals or metal compounds of Group IIa, IIIb, IVb, Vib or VIIb.

19. The catalyst or catalyst precursor of claim 18, wherein the metal or metal compound of Group IIa, IIIb, IVb, VIb or VIIb comprises manganese or zirconium oxide.

20. The catalyst or catalyst precursor of claim 15, further comprising one or more additional metals of Group VIIb or VIII.

21. The catalyst or catalyst precursor of claim 20, wherein the additional metal of Group VIIb or VIII is selected from the group consisting of rhenium, platinum and palladium.

22. The catalyst or catalyst precursor of claim 15, wherein the carrier is formed by a process selected from the group consisting of spray-drying, pressing and extruding.

23. The catalyst or catalyst precursor of claim 15, wherein one or more metal salts is added to the carrier by impregnation or deposition precipitation, followed by drying and calcination.

24. A process for the preparation of a catalyst or a catalyst precursor comprising:
mixing crystalline titania in which at least 50 wt %/o of the crystalline titania is present as brookite, a liquid, and a Group VIII containing compound, which is at least partially insoluble in the amount of liquid used, to form a mixture;
shaping and drying the mixture; and
calcining the mixture.

25. The process for the preparation of a catalyst or a catalyst precursor of claim 24, wherein the Group VIII compound is selected from the group consisting of metallic cobalt containing compounds, cobalt hydroxide containing compounds and cobalt oxide.

26. An activated catalyst, comprising a Group VIII metal or a Group VIII metal compound and a shaped carrier containing crystalline titania, wherein at least 50 wt % of the crystalline titania is present as brookite and wherein the carrier comprises between 40 and 100 wt % of crystalline titania based on the total weight of the carrier, the shaped catalyst carrier not being a coated catalyst carrier and wherein the catalyst has been reduced with hydrogen at an elevated temperature.

27. A process for the preparation of hydrocarbons comprising contacting a mixture of carbon monoxide and hydrogen with an activated catalyst, comprising a Group VIII metal or a Group VIII metal compound and a shaped carrier containing crystalline titania, wherein at least 50 wt % of the crystalline titania is present as brookite and wherein the carrier comprises between 40 and 100 wt % of crystalline titania based on the total weight of the carrier, the shaped catalyst carrier not being a coated catalyst carrier and wherein the catalyst has been reduced with hydrogen at an elevated temperature.

28. The process of claim 27, further comprising hydroisomerisation/hydrocracking of the hydrocarbon to produce a middle distillate product.
